(19) United States
(12) Patent Application Publication
(10) Pub. No.: US 2014/0200464 A1
(43) Pub. Date: Jul. 17, 2014

(54) PATIENT TEMPERATURE CHANGE COMBINED WITH REMOTE ISCHEMIC CONDITIONING

(71) Applicant: Physio-Control, Inc., Redmond, WA (US)

(72) Inventors: Brian Daniel Webster, Sammamish, WA (US); Daniel Ralph Piha, Bellevue, WA (US); Fred W. Chapman, Newcastle, WA (US)

(73) Assignee: Physio-Control, Inc., Redmond, WA (US)

(21) Appl. No.: 14/149,534

(22) Filed: Jan. 7, 2014

Related U.S. Application Data
(60) Provisional application No. 61/753,166, filed on Jan. 16, 2013.
(60) Provisional application No. 61/755,408, filed on Jan. 22, 2013.

(51) Int. Cl. A61H 9/00; A61B 5/021

(52) U.S. Cl. CPC A61H 9/0007; A61B 5/021
USPC 600/485; 601/150

(57) ABSTRACT

A single system may provide to a patient: temperature change, remote ischemic conditioning, and sometimes both concurrently. The system may include a patient unit that includes an inflatable bladder, and a duct having a cavity. The patient unit is intended to be applied around a patient's limb. A temperature subsystem can force a flow of a first fluid through the cavity so that the first fluid can exchange heat with the patient's limb. The pressure subsystem may force a fluid into the bladder, to establish pressure against the limb. A controller may control both the temperature subsystem and the pressure subsystem, so as to control the treatment received by the patient.

### CROSS REFERENCE TO RELATED PATENT APPLICATIONS

This patent application claims priority from U.S. Provisional Patent Application Ser. No. 61/753,166, filed on Jan. 16, 2013, titled: “DEVICE TO ASSIST WITH DELIVERY OF REMOTE ISCHEMIC CONDITIONING THERAPY”, the disclosure of which is hereby incorporated by reference for all purposes.

This patent application claims priority from U.S. Provisional Patent Application Ser. No. 61/755,408, filed on Jan. 22, 2013, titled: “ISCHEMIC CONDITIONING IN CONJUNCTION WITH CHEST COOLING AND COMPRESSION”, the disclosure of which is hereby incorporated by reference for all purposes.

### BACKGROUND

In some instances, cardiac patients can benefit from receiving ischemic conditioning (IC) treatment. The treatment can be provided by a system that induces transient ischemia to a limb of the patient, by occluding blood circulation and then releasing the limb. Sometimes, there are repeated cycles of occluding and releasing.

Ischemic conditioning (IC) treatment invokes a protective mechanism inherent in the biology of humans and other species. The protective mechanism reduces the extent of injury caused by tissue ischemia, and of injury caused by reperfusion of that tissue after the period of ischemia, in other parts of the body, by a cause such as heart attack. The limb is typically remote from the other part of the body, such as the heart, and that is why this treatment can also be called remote ischemic conditioning (rIC).

Ischemic conditioning treatment can be provided before, during, or after other the period when the tissue of concern is ischemic. In such instances, the treatment can be called, more particularly, ischemic preconditioning, ischemic conditioning, and ischemic post-conditioning, respectively. In this document, “ischemic conditioning” can mean treatment before, during, or after the ischemic event of the tissue of concern.

More is taught in U.S. Pat. No. 7,717,855 B2 plus US patent applications 2010/0324429 A1, 2013/0184745 A1 and 2013/0218196 A1, all of which are incorporated by reference.

In some instances, cardiac patients can benefit from being cooled. Cooling can be provided by dressing the body with cold suits, as is taught in U.S. Pat. No. 7,179,279 B2 which are incorporated by reference. There are also devices for cooling non-cardiac patients, such as trauma patients, for example as is taught in U.S. Pat. No. 7,896,910, plus US patent applications 2006/0191675 A1, 2010/0139294 A1 and 2011/0098792 A1, all of which are incorporated by reference. In some instances the cooling is topical, such as to a knee, elbow or limb of the patient. Devices that cool only a limb may even apply some pressure against it, so as to improve the thermal contact and thus increase the rate at which heat is exchanged. As these devices are applied for a long time, the pressure may not be enough to cause ischemia.

### BRIEF SUMMARY

The present description gives instances of systems, controllers for systems and methods, the use of which may help overcome problems and limitations of the prior art.

In embodiments, a single system may provide to a patient; temperature change, remote ischemic conditioning, and sometimes both concurrently. The system may include a patient unit that includes an inflatable bladder, and a duct having a cavity. The patient unit is intended to be applied around a patient's limb. A temperature subsystem can force a flow of a first fluid through the cavity so that the first fluid can exchange heat with the patient's limb. The pressure subsystem may force a fluid into the bladder, to establish pressure against the limb. A controller may control both the temperature subsystem and the pressure subsystem, so as to control the treatment received by the patient.

An advantage over the prior art is that embodiments of a single system can be used for the two separate functions of changing the temperature and remote ischemic conditioning. Another advantage is that embodiments can provide these two functions concurrently, while occupying only one limb of the patient.

These and other features and advantages of this description will become more readily apparent from the following Detailed Description, which proceeds with reference to the drawings, in which:

### BRIEF DESCRIPTION OF THE DRAWINGS

FIG. 1 is a diagram of a sample TRICON system made according to embodiments of the invention.

FIG. 2 is a diagram of a detail of a sample patient unit for a TRICON system, made according to an embodiment.

FIG. 3 is a block diagram of a sample controller for a TRICON system, made according to embodiments.

FIG. 4 is a diagram of a sample user interface for a TRICON system, made according to embodiments.

FIG. 5 is a diagram of sample salient parts of the TRICON system of FIG. 1 that operate when a dial of FIG. 4 points to a second indication according to an embodiment.

FIG. 6 is a diagram of sample salient parts of the TRICON system of FIG. 1 that operate when a dial of FIG. 4 points to a third indication according to an embodiment.

FIG. 7 is a diagram of sample salient parts of the TRICON system of FIG. 1 that operate when a dial of FIG. 4 points to the third indication according to a different embodiment.

FIG. 8 is a diagram of sample salient parts of the TRICON system of FIG. 1 that operate when a dial of FIG. 4 points to a fourth indication according to an embodiment.

FIG. 9 is a diagram of a sample TRICON system made according to embodiments where it is controlled by a cooperating device.

FIG. 10 is a flowchart for illustrating methods according to embodiments.

FIG. 11 is a table for showing different time frames, with reference to ischemic conditioning, for which a TRICON system according to embodiments can be applied.

### DETAILED DESCRIPTION

As has been mentioned, the present description is about providing temperature change and remote ischemic conditioning to a patient by a single system. Embodiments are now described in more detail.

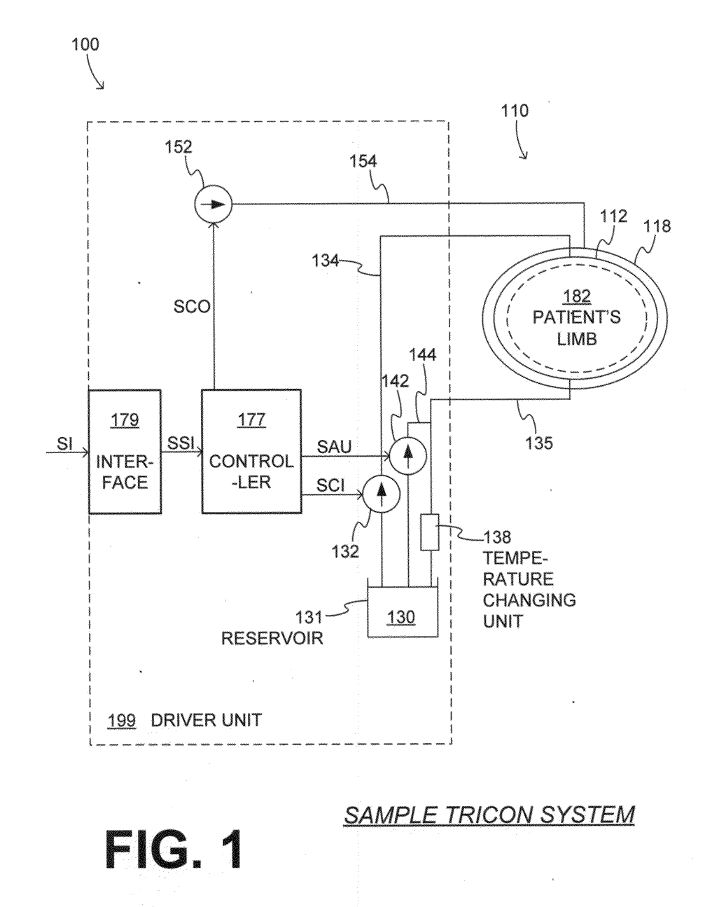

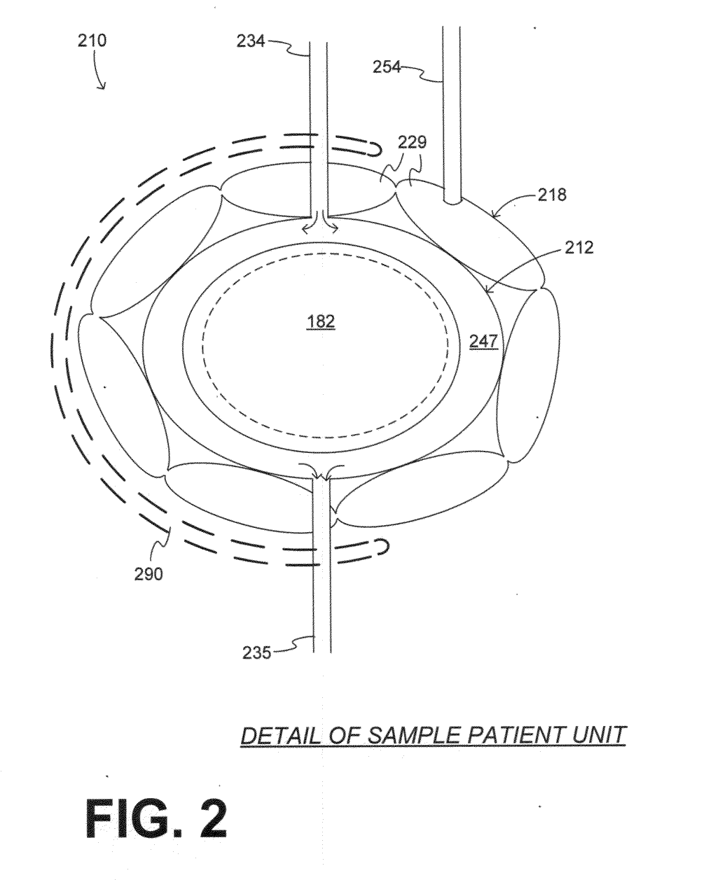

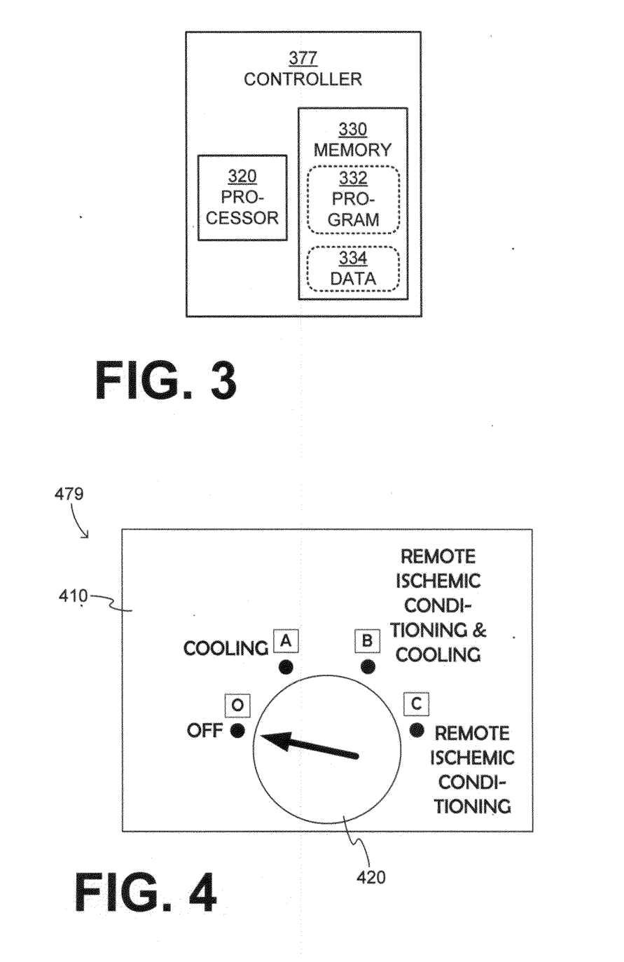

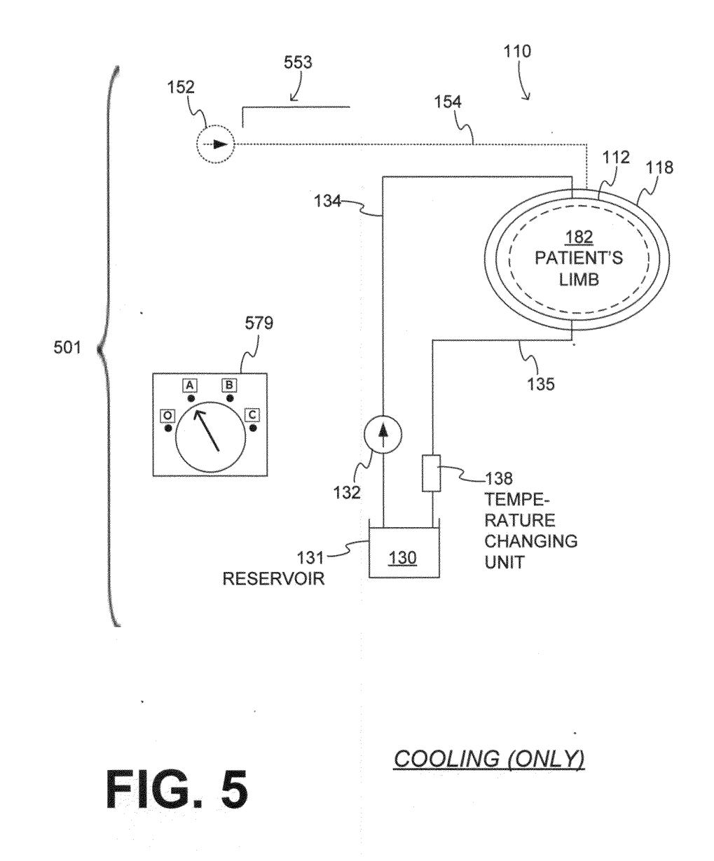

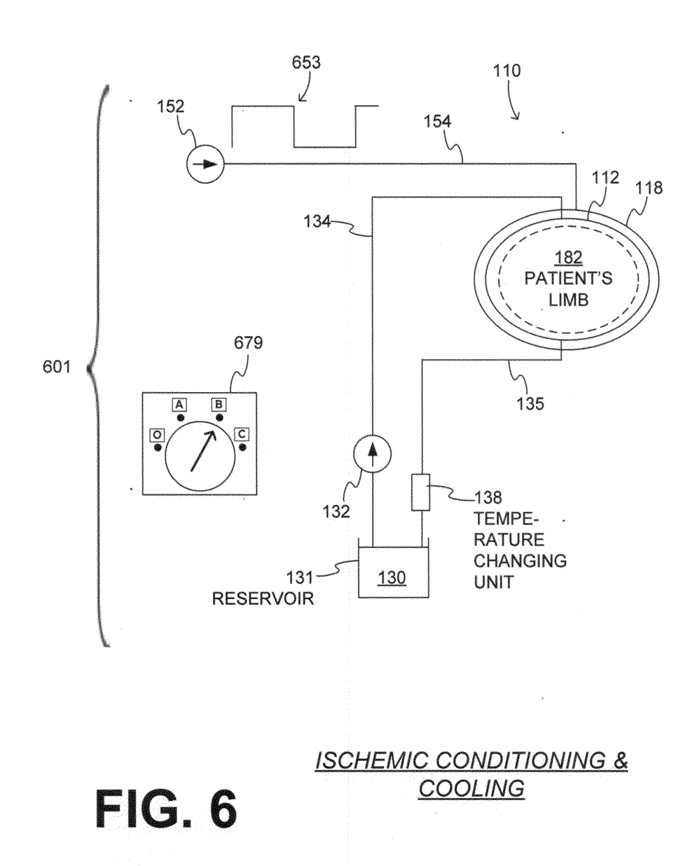

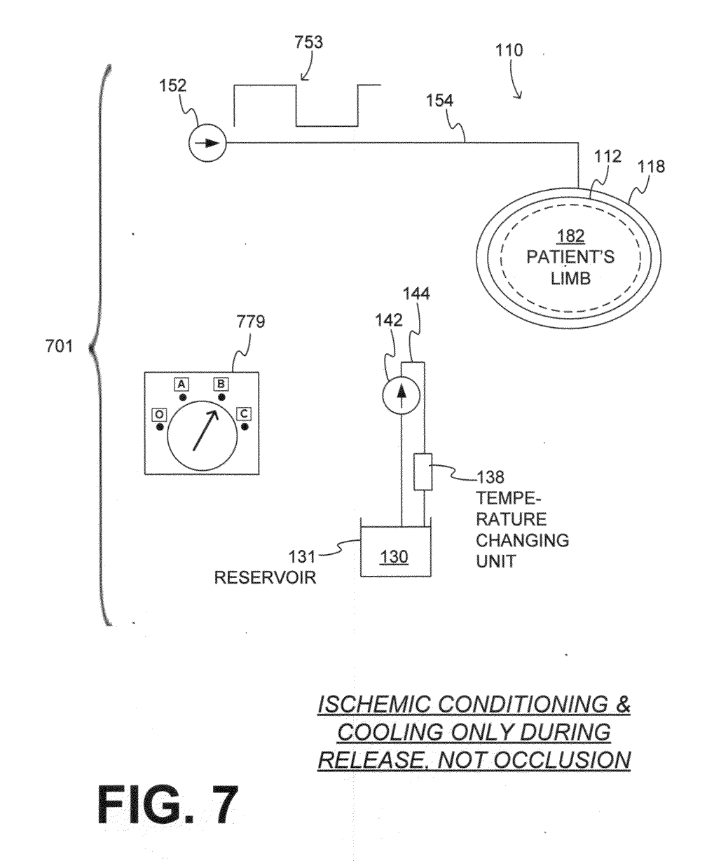

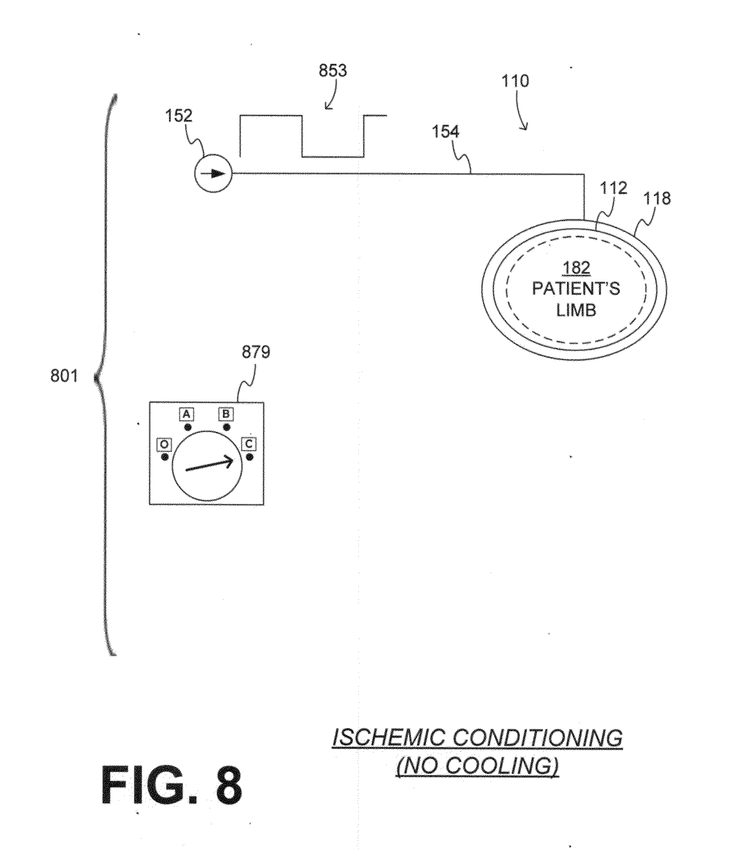

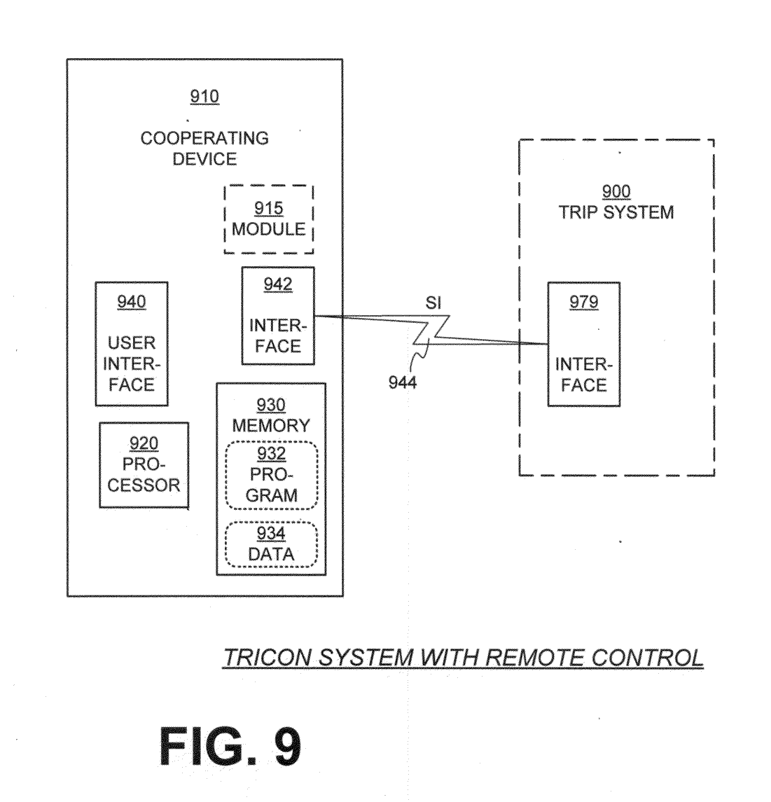

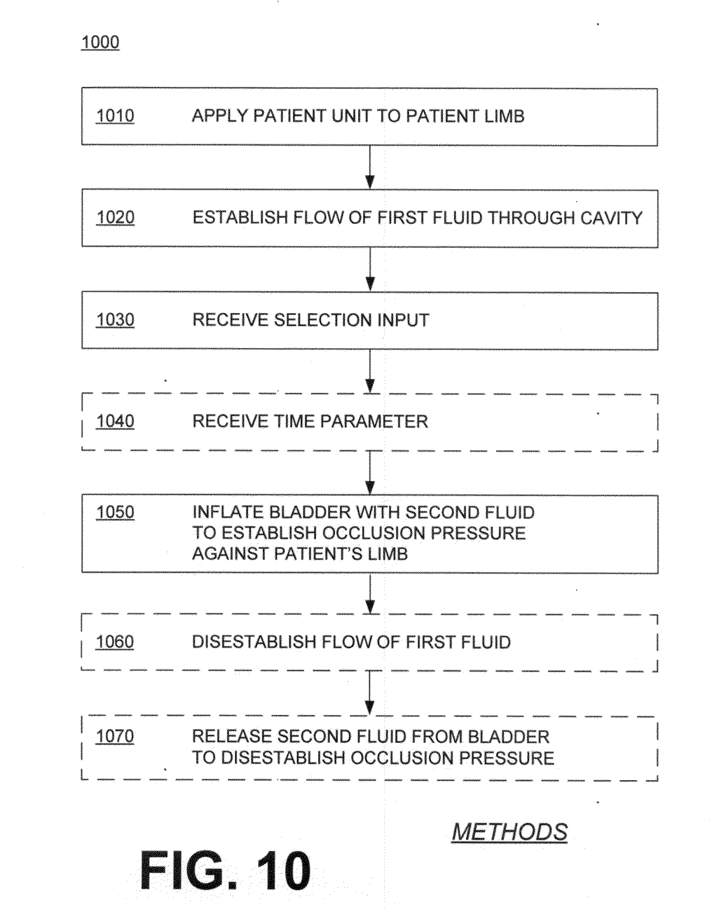

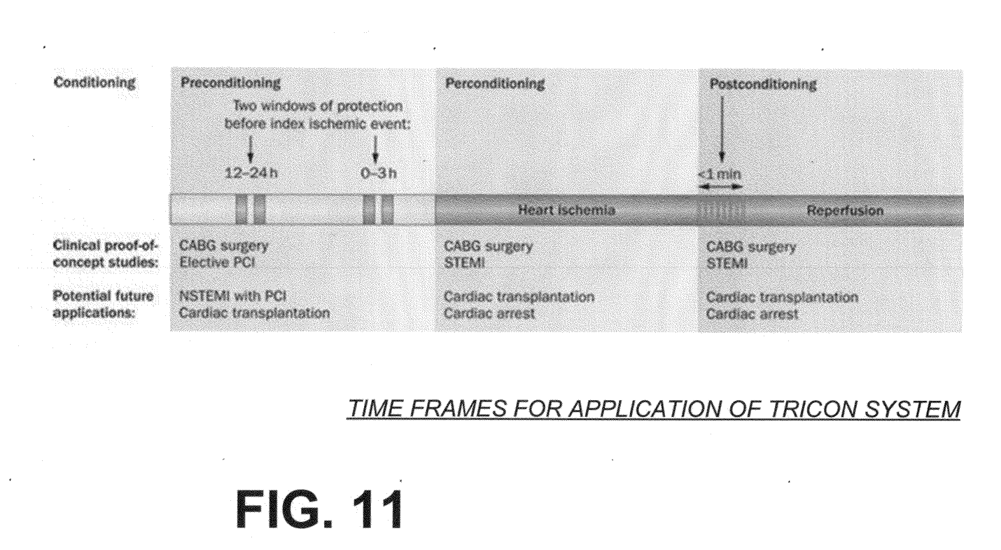

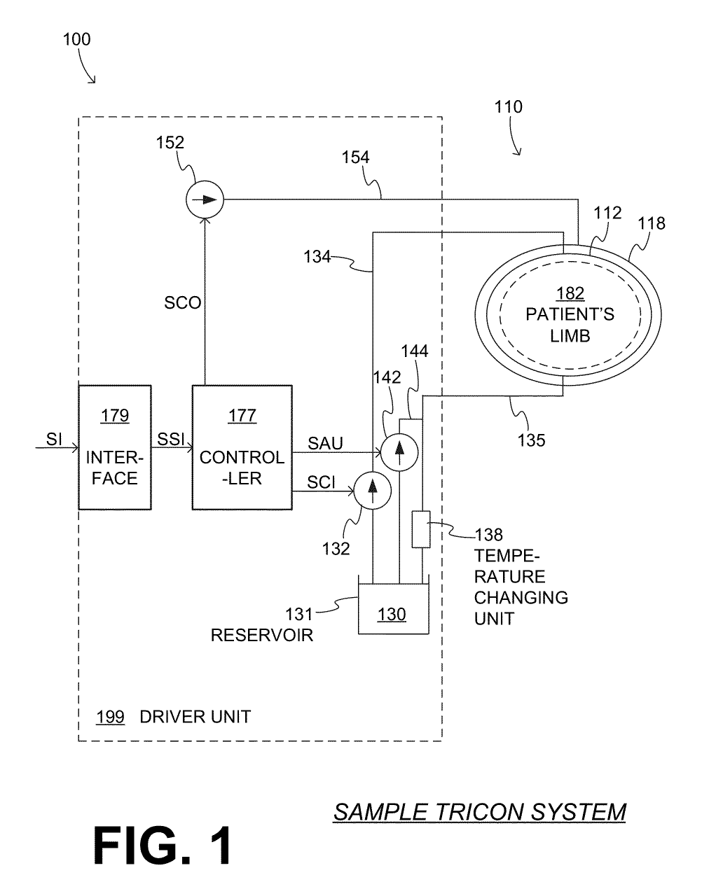

FIG. 1 is a diagram of a sample TRICON system 100, which is made according to embodiments. The acronym TRICON is derived from changing temperature and remote ischemic conditioning.

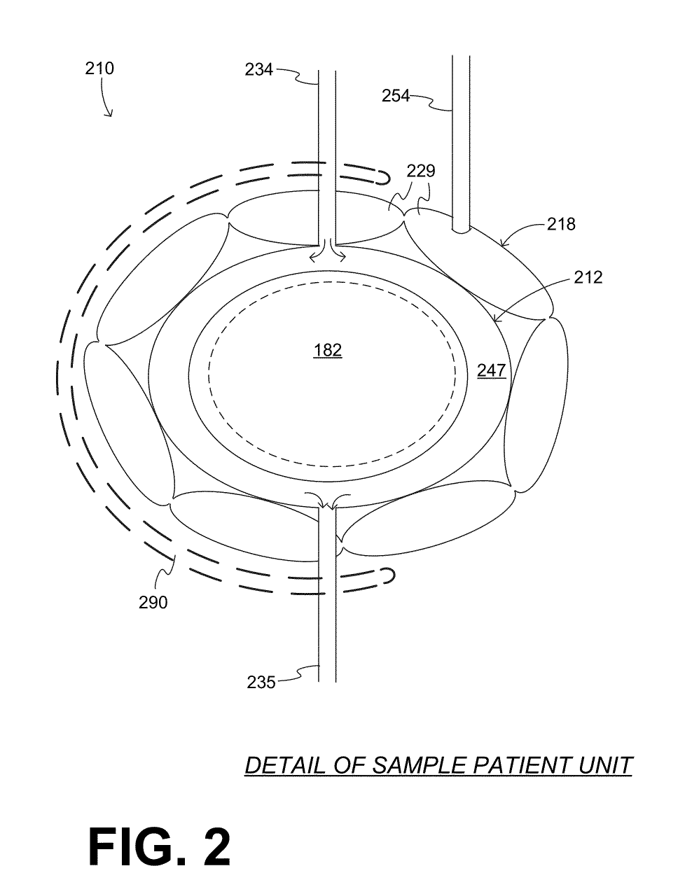

TRICON system 100 includes a patient unit 110. Patient unit 110 is configured to be attached to a patient's limb 182, and is intended to deliver temperature change plus pressure to limb 182. For delivering pressure, patient unit 110 includes an inflatable bladder 118. For delivering temperature change, patient unit 110 includes a flexible duct 112. Duct 112 has a cavity for circulating fluid, as will be understood later from the description of FIG. 2.

Still referring to FIG. 1, in some embodiments, bladder 118 is coupled to duct 112. Coupling can be permanent, or the components of patient unit 110 can be modular, for example where one is inserted in the other and so on. In some embodiments, coupling is such that, when patient unit 110 is attached to patient's limb 182, duct 112 contacts limb 182, and bladder 118 substantially surrounds duct 112.

System 100 includes a temperature subsystem for changing the patient temperature, via patient unit 110. System 100 also includes a pressure subsystem, for exerting pressure on limb 182, via patient unit 110. The individual components of the temperature subsystem and the pressure subsystem are shown in FIG. 1, but these components are not grouped in terms of their subsystems, so as not to obscure the drawing.

The temperature subsystem can be used make the patient temperature higher or lower. The temperature is changed at the patient's limb, starting from the skin and continuing inward. Once it reaches the vascular system, the blood temperature is also changed and, when the blood is circulating, heat is removed or added also to the other parts of the body that the blood circulates to. For a cardiac patient, the temperature change of main interest is cooling, so as to prevent damage to the patient, but heating can equivalently be applied.

The temperature subsystem includes a reservoir 131. Reservoir 131 is configured to contain a first fluid 130 that will effectuate heat transfer for changing the patient temperature. First fluid 130 can be a liquid such as water, or any other fluid as is known to the person skilled in the art.

In system 100, the temperature subsystem also includes a circulation pump 132, and at least one hose. In this embodiment, two hoses 134, 135 are provided. In the example of FIG. 1, circulation pump 132 and hoses 134, 135 are coupled to establish a circular flow of the first fluid from reservoir 131 through the cavity of duct 112.

The temperature subsystem moreover may include a temperature changing unit 138. Unit 138 is configured to change a temperature of first fluid 130, as it flows through hose 135. This way, when first fluid 130 reaches the cavity of duct 112, it exchanges heat with patient limb 182 according to the effect of temperature changing unit 138. In some embodiments, unit 138 may surround hose 135. In other embodiments, unit 138 may provide its own internal path, and two pieces of hose 135 can be coupled to the ends of the internal path. Again, for a cardiac patient, temperature changing unit 138 can be a cooling unit, while in other embodiments it can be a heating unit.

As will be seen, in some modes of operation, circulation pump 132 may stop operating, and the fluid may stop circulating through the cavity of duct 112. Still, the effect of the temperature changing unit 138 may persist, for example because unit 138 might not be turned off quickly enough, or it might be turned on again soon. So, it may be desirable to continue circulating first fluid 130 through temperature changing unit 138, so that first fluid 130 does not freeze in the internal path, or become too hot in a single location. Accordingly, in some embodiments, system 100 includes an auxiliary pump 142 and at least one hose 144. Auxiliary pump 142 and hose 144 can be coupled to establish an auxiliary circular flow of first fluid 130 from reservoir 131 through temperature changing unit 138. In some embodiments, this circular flow bypasses the cavity of duct 112, which might have no flow through it at the time.

The temperature subsystem may also have other components, such as a pressure gauge, one or more valves, feedback control of circulation pump 132 and auxiliary pump 142, a thermometer, feedback control of temperature changing unit 138, and so on.

Moreover, the above-mentioned pressure subsystem may include a compression pump 152 and at least one hose 154. In the example of FIG. 1, compression pump 152 and hose 154 are coupled to inflate the bladder with a second fluid, so as to establish a pressure against patient's limb 182.

The pressure subsystem may also have other components, such as one or more valves, feedback control of compression pump 152, and so on. The other components could also include a pressure gauge, configured to measure the pressure against the patient's limb, such as the pressure of the second fluid that is inflating the bladder. The other components could further include a sensor for measuring the patient's blood pressure non-invasively. For example the sensor could be a sound sensor, and operate in combination with the above-mentioned pressure gauge. The patient's blood pressure could be measured from sounds while the pressure is increasing and/or decreasing, and more particularly from an output of the pressure gauge at a time indicated by an output of the sound sensor. The measured blood pressure could be displayed at a user interface. Additionally, a record of the measured blood pressure could be stored in a memory or exported to another device, such as is described later in this document.

Optionally, system 100 additionally includes a driver unit 199. Driver unit 199 contains circulation pump 132 and compression pump 152, and can be implemented in many ways for that effect. Driver unit 199 can also advantageously include other components of system 100. In addition, a single power supply may be optionally provided for system 100, which is configured to power circulation pump 132 and compression pump 152.

System 100 may further include an interface 179. Interface 179 can be configured to receive a selection input SI, which is related to what system 100 is expected to do. Selection input SI may be received in any number of ways, and interface 179 can be constructed accordingly. For example, interface 179 can be a user interface, and configured to be operated by a rescuer for receiving the selection input. For another example, interface 179 can be automatic, and configured to receive the selection input from another, cooperating device, as will be seen later in this document.

TRICON system 100 may further include a controller 177. Controller 177 can be configured to control circulation pump 132 by a signal SCI, and compression pump 152 by a signal SCO. Moreover, controller 177 may control additional elements of system 100. For example, in embodiments where auxiliary pump 142 is also provided, controller 177 can be configured to also control auxiliary pump 142 by a signal SAU.

Controller 177 may be able to control pumps 132, 152 so that they operate in any one of a number of desirable modes. The mode can be selected according to selection input SI, which can be learned by decoding a signal SSI that controller 177 may receive from interface 179. A number of modes of operation are possible for pumps 132, 152, for system 100 to perform its desired tasks, as will be described later.

FIG. 2 is a diagram of a detail of a sample patient unit 210, made according to embodiments. Patient unit 210 could be patient unit 110 of FIG. 1.

Patient unit 210 includes a duct 212, which is wrapped around limb 182 of the patient. Preferably duct 212 makes good thermal contact with limb 182, and such is not shown only so that the various elements in FIG. 2 could be illustrated individually more clearly. Patient unit 210 includes a cavity 247. Two hoses 234, 235, which are akin to hoses 134, 135 of FIG. 1, communicate with cavity 247. The first fluid flows via hose 234 to cavity 247, and returns via hose 235.

Patient unit 210 also includes a bladder 218, which is coupled with duct 212. Bladder 218 can be made in a number of ways, such as the cuff that is wrapped around a person's arm for non-invasively measuring their blood pressure. In the example of FIG. 2, bladder 218 is made of a series of chambers 229 whose interiors communicate. A hose 254, akin to hose 154, can inflate chambers 229 more than is shown, so as to establish pressure around limb 182. More inflating can result in more pressure, such as occlusion pressure or a little higher than occlusion pressure.

In some embodiments, patient unit 210 further includes a bracket 290, which may surround bladder 218 at least partly. When inflated, bladder 218 may thus establish the pressure by being at least partly within bracket 290, and bracing against bracket 290.

Referring now to both FIG. 1 and FIG. 2, a number of modes of operation for system 100 are now described.

In a first mode, system 100 may perform temperature change, such as cooling or heating. In this first mode, circulation pump 132 establishes the above-mentioned circular flow of first fluid 130 through the cavity of duct 112. In FIG. 2, the direction of the flow of the first fluid within cavity 247 is shown by arrows.

In addition, in the first mode, the pressure from bladder 218 on limb 182 may be nil, for example by not inflating bladder 218. Alternately, compression pump 152 may optionally establish some pressure via bladder 118, or 218. Such a pressure may be desirable for duct 112 to make good thermal contact with limb 182, for heat exchange to happen more efficiently. However, the pressure in the first mode might not be much larger beyond that, so as to not impact the patient's blood circulation—after all, the temperature change may be performed for a long time. So, the pressure in the first mode might be less than an occlusion pressure, which would have been strong enough to cut off the blood circulation.

For another example, in a second mode, system 100 may perform remote ischemic conditioning. In the second mode, the pressure created by compression pump 152 is an occlusion pressure, i.e. a pressure that is large enough to substantially occlude blood circulation through the patient's limb 182. The occlusion pressure will be maintained for some time, and then released to allow reperfusion. The whole process may be repeated, in cycles of occlusions and releases.

During the second mode, circulation of first fluid 130 through the cavity of duct 112 may continue at the same rate as in the first mode, slow down, or become disestablished. The design decision will be made based on one or more factors, as will be evident to a person skilled in the art. For example, one factor is whether the compression from bladder 218 pinches duct 212 too much to permit flow at the same rate as in the first mode. The effect of pinching may be ameliorated by including spacers within cavity 247, in which case duct 212 contributes to the pressure being an occlusion pressure. Another factor can be the reality that blood does not circulate during occlusion, and therefore does not effectuate temperature change in places of the body other than the limb 182.

It will be appreciated that these first and second modes are not mutually exclusive. More modes can be devised. For example, as seen above in the second mode temperature change can be performed concurrently with remote ischemic conditioning. More detail will be provided later in this document.

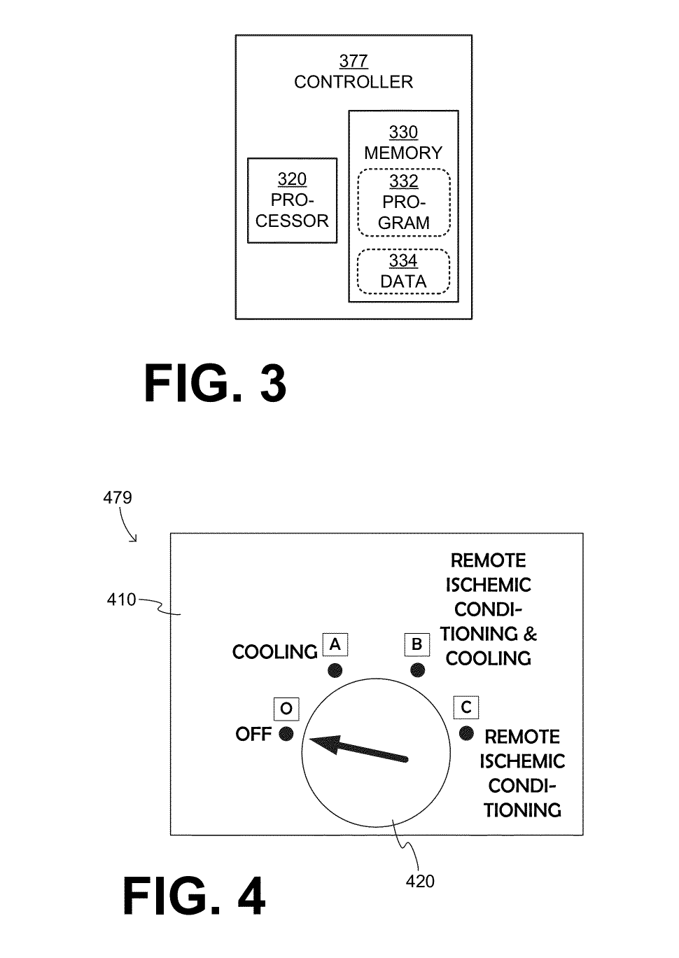

FIG. 3 is a block diagram of a sample controller 377, made according to embodiments. Controller 377 could be controller 177 of FIG. 1. In this embodiment, controller 377 includes a processor 320, but it can also be more than one processors. Processor 320 may be implemented as a Central Processing Unit (CPU), a digital signal processor, a microprocessor, a microcontroller, an application-specific integrated circuit (ASIC), a programmable logic device (PLD), or other implementation. Controller 377 can be optionally combined in a single chip with a memory controller and a peripherals interface.

Controller 377 also includes a memory 330, which can include both persistent/non-volatile and non-persistent/volatile memory components. Memory 330 can be implemented in any technology for memory for such devices, volatile memory, non-volatile memory (NVM), for example RAM, ROM, EEPROM, flash memory, and so on. As such, memory 330 can include a non-transitory computer-readable storage medium. Memory 330 can store a program 332 and data 334. Program 332 may include instructions in the form of code that can be read and executed by controller 377. Executing is performed by physical manipulations of physical quantities, and may result in the functions, processes, actions and/or methods to be performed, and/or controller 377 to cause other devices or components or blocks to perform such functions, processes, actions and/or methods.

FIG. 4 is a diagram of a sample user interface 479, made according to embodiments. User interface 479 could be interface 179 of the system of FIG. 1. User interface 479 can be implemented for embodiments where the temperature change is cooling. Other embodiments can be for where the temperature change is heating, or where it is possible to alternate between heating and cooling.

User interface 479 includes a panel 410. If implemented for the example of FIG. 1, panel 410 could be on driver unit 199. Panel 410 includes indications “0”, “A”, “B”, “C”, with further writing as to what each indication means. User interface 479 also includes a dial 420 with an arrow. Dial 420 can be rotated by the rescuer, so that the arrow can point to the desired indication in panel 410. It will be recognized that, in this example, the four indications correspond to four modes of operation for the system. One of the modes is OFF, and the system is powered off.

Of course, a user interface such as user interface 479 can provide more features. For example, it can include some sort of display that shows patient physiological parameters, such as the patient's vital signs. The vital signs could include the patient's temperature measured at a location near where patient unit 110 is applied, and also possibly at additional locations for reference. The vital signs could further include the patient's blood pressure, which could be measured non-invasively as per the above. The display could also show progress of the treatment, such as progress within a therapy cycle and progress within a configured preferable number of therapy cycles.

A user interface according to embodiments could provide even more features. For example, it could further include an interface that allows the rescuer, or some other authorized person like a medical director, to configure the duration of the ischemia period or cycle, the duration of the release/reperfusion period of cycle, and a prompting behavior, including how many cycles to prompt for. Moreover, it can include an alert/reminder system that provides visible and/or audio prompts at the end of a release cycle, and a reminder to the rescuer that it is acceptable to initiate the next cycle.

As this is contemplated, there are tradeoffs between automating the delivery of rIC and not. Field rescuers might appreciate some level of automation, facilitating administration of the therapy in a context where they are very busy and otherwise preoccupied. On the other hand, emergency care of heart attack and stroke patients has many real world logistic complications, and it may be undesirable to fully automate rIC therapy. A semi-automatic approach could be preferable, or an adjustment from automating the delivery to not doing so. This allows more flexibility in the delivery of the rIC therapy that would not be possible with a fully-automatic RIC device. Cycles could be initiated at more opportune moments during the care and management of the patient, with duration of the intervals between the ischemic or compression periods being adjusted at the discretion of the paramedic (or other care provider), possibly informed by measurements taken during those release intervals.

The modes of the example of FIG. 4 are now described in more detail.

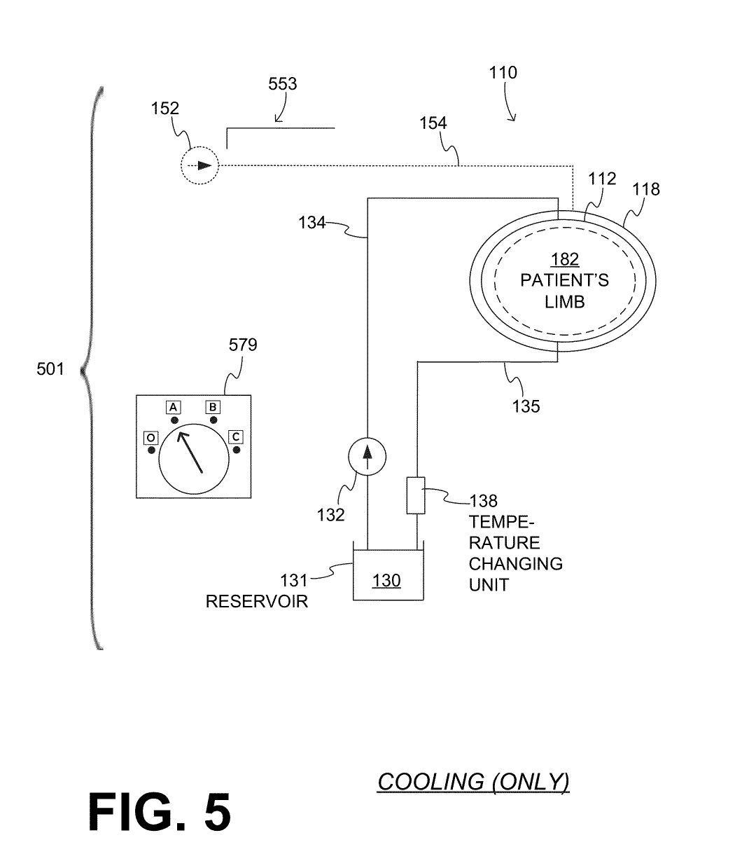

FIG. 5 is a diagram of sample salient parts 501 of TRICON system 100 of FIG. 1 that operate according to an embodiment. A box 579 shows the dial of FIG. 4 pointing to second indication “A”, where only cooling is performed. Circulation pump 132 is operating, along with temperature changing unit 138. Compression pump 152 operates only optionally. If it does, it is to establish a pressure large enough for good thermal contact, but not to effectuate occlusion of limb 182; the constant pressure it builds is shown in a waveform segment 553.

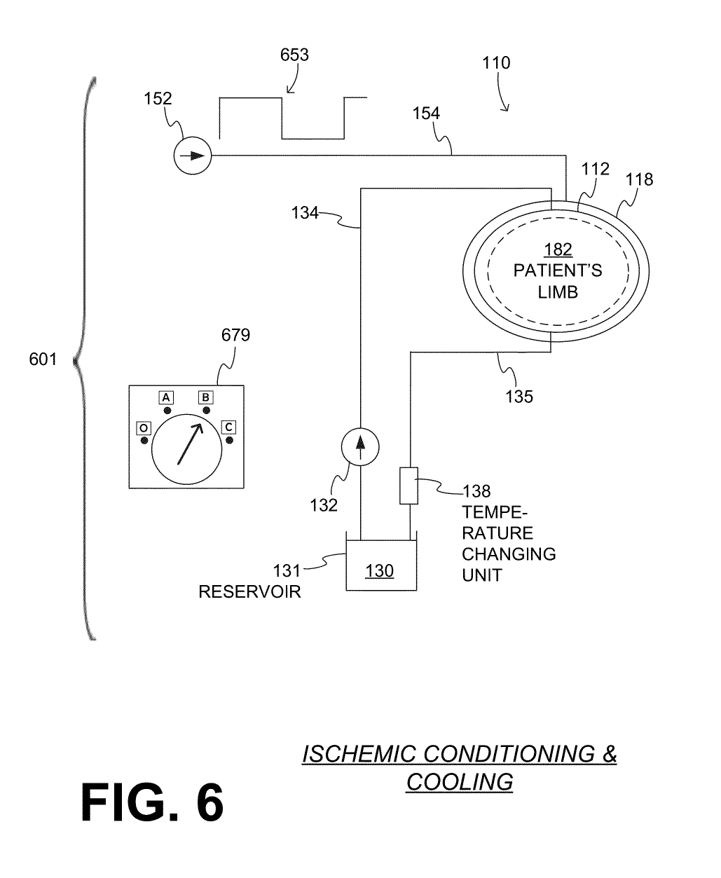

FIG. 6 is a diagram of sample salient parts 601 of TRICON system 100 of FIG. 1 that operate according to an embodiment. A box 679 shows the dial of FIG. 4 pointing to third indication “B”, where cooling is performed concurrently with remote ischemic conditioning. Circulation pump 132 is operating, along with temperature changing unit 138. Plus, compression pump 152 operates to effectuate cycles of occlusions alternating with cycles of releases of limb 182, as seen in a waveform segment 653. The pressure reached is higher than in FIG. 6, because it achieves occlusion. During releases, the pressure drops down to zero, or to the same value as in waveform segment 553, and blood circulation is restored. The instant of FIG. 6 is during a cycle of either occlusion or release, and circulation pump 132 could be operating in both types of cycles.

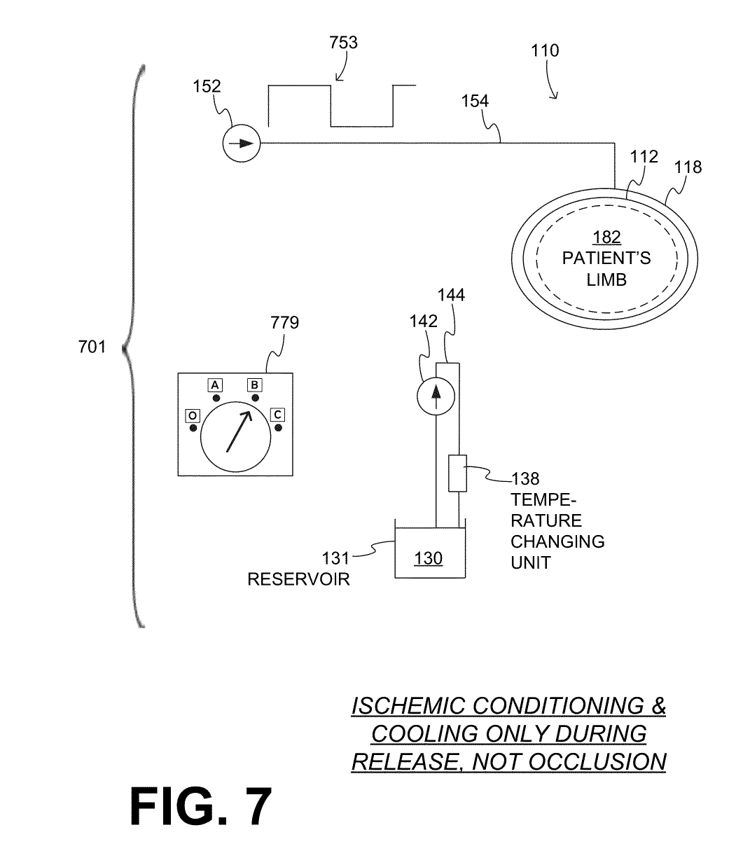

FIG. 7 is a diagram of sample salient parts 701 of TRICON system 100 of FIG. 1 that operate according to a different embodiment. A box 779 shows the dial of FIG. 4 pointing to third indication “B”, similarly with FIG. 6. Compression pump 152 operates to effectuate cycles of occlusions alternating with cycles of releases of limb 182, as seen in a waveform segment 753 that is similar to waveform segment 653. In the embodiment of FIG. 7, circulation takes place only during one or more cycles of releases, but not during cycles of occlusion. The instant of FIG. 7 is during a cycle of occlusion. Circulation pump 132 is not operating, but auxiliary pump 142 is operating to drive the first fluid through temperature changing unit 138 via hose 144. A cycle of release for the embodiment of FIG. 7 is not shown in FIG. 7, but happens to be the same as in FIG. 6.

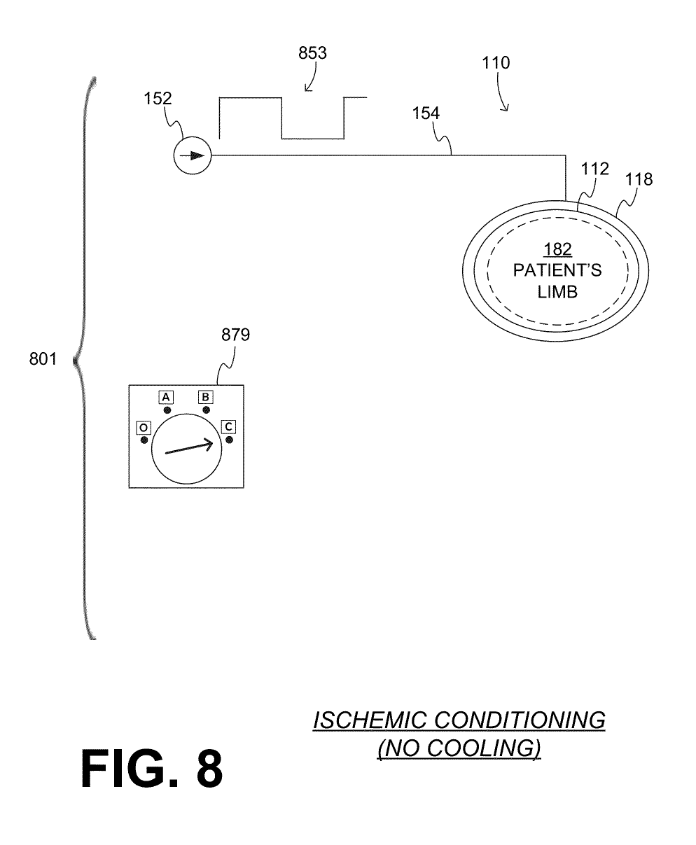

FIG. 8 is a diagram of sample salient parts 801 of TRICON system 100 of FIG. 1 that operate according to an embodiment. A box 879 shows the dial of FIG. 4 pointing to fourth indication “C”, where only remote ischemic conditioning is performed. Compression pump 152 operates to effectuate cycles of occlusions alternating with cycles of releases of limb 182, as seen in a waveform segment 853 that is similar to waveform segment 653. Neither circulation pump 132 nor temperature changing unit 138 are shown in FIG. 8, because neither needs to be operating.

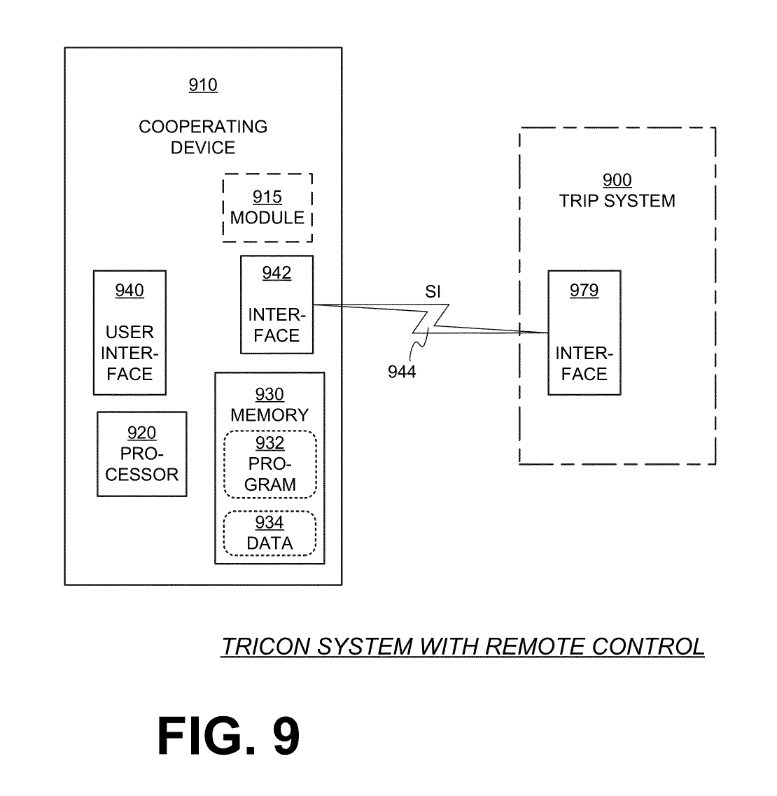

FIG. 9 is a diagram of a sample TRICON system 900 made according to embodiments. System 900 has components as described with reference to FIG. 1, including interface 979 that is an automatic version of interface 179 of FIG. 1.

TRICON system 900 can be controlled by a cooperating device 910. More particularly, interface 979 can establish a communication link 944 with cooperating device 910. Link 944 can be wireless, as shown, or wired. A signal can be communicated over link 944, which communicates selection input SI. This way, cooperating device 910 can instruct system 900 when to cool the patient, when to perform remote ischemic conditioning, and so on. The feature is particularly useful when cooperating device 910 is a medical device, like a defibrillator, a chest compression machine, etc., and it can coordinate a treatment that it administers with a treatment that TRICON system 900 administers. All this is possible also if system 900 is not a full TRICON system but either only a cooling device, or a compressing device.

Embodiments of cooperating device 910 are now described in detail. Device 910 can be for example, a medical device by optionally including a module 915. Module 915 can be a defibrillator, a chest compression machine, a monitor of a patient parameter, and so on. Alternately, device 910 can be a portable electronic device, such as a tablet computer, a smartphone, and so on.

Device 910 has a processor 920 that can be made as processor 320. It also has a memory 930 that can be made as memory 330. Memory 930 can store one or more programs 932 and data 934, similarly with memory 330.

Device 910 also has an interface 942 that can support communication link 944. For example, if link 944 is wireless, interface 942 and interface 979 include antennas. Else, if link 944 is wired, then interface 942 and interface 979 may include plugs, a wire, and so on.

Device 910 also has a user interface 940, which can be used by an operator, for example a rescuer. The operator can enter commands that amount to selection signal SI directly, or to command that the selection signal be looked up from data 934. Importantly, along with selecting the mode, selection signal can also carry one or more parameters for operation of TRICON system 900. One such parameter can be a time parameter, such as a duration of the occlusion cycles, a duration of the release cycles, and so on. The parameter can also be passed along link 944, and be obeyed by system 900. In other words, the pressure in the above-described second mode can be established according to the time parameter.

Additionally, TRICON system 900 can also pass information to cooperating device 910. Such information can include known status of the patient, history of service to the patient, status of system 900, and so on. Such information can become known to the operator via user interface 940.

Cooperating device 910 may also have additional features. For example, via interface 940 it can provide the same information and enable the same functionality as interface 179. Plus, it can also provide advisory recommendations to the rescuer to consider effectuating temperature change or delivering rIC therapy, recommendations that are delivered when the monitor/defibrillator detects conditions suggesting ischemic emergencies. For example, a 12-lead interpretive algorithm, upon issuing a statement indicating ST-elevated myocardial Infarction (such as “meets STEMI criteria”), also issues a recommendation to initiate rIC therapy when possible. The recommendation can be issued before even TRICON system 900 has been brought to the scene, or device 910 has detected its presence.

Moreover, methods and algorithms are described below. These methods and algorithms are not necessarily inherently associated with any particular logic device or other apparatus. Rather, they are advantageously implemented by programs for use by a computing machine, such as a general-purpose computer, a special purpose computer, a microprocessor, etc.

Often, for the sake of convenience only, it is preferred to implement and describe a program as various interconnected distinct software modules or features, individually and collectively also known as software. This is not necessary, however, and there may be cases where modules are equivalently aggregated into a single program, even with unclear boundaries. In some instances, software is combined with hardware, in a mix called firmware. Software or firmware according to embodiments can cause controller 177, processor 320, and/or processor 920 to perform the methods of the invention.

This detailed description includes flowcharts, display images, algorithms, and symbolic representations of program operations within at least one computer readable medium. An economy is achieved in that a single set of flowcharts is used to describe both programs, and also methods. So, while flowcharts described methods in terms of boxes, they also concurrently describe programs.

Methods are now described.

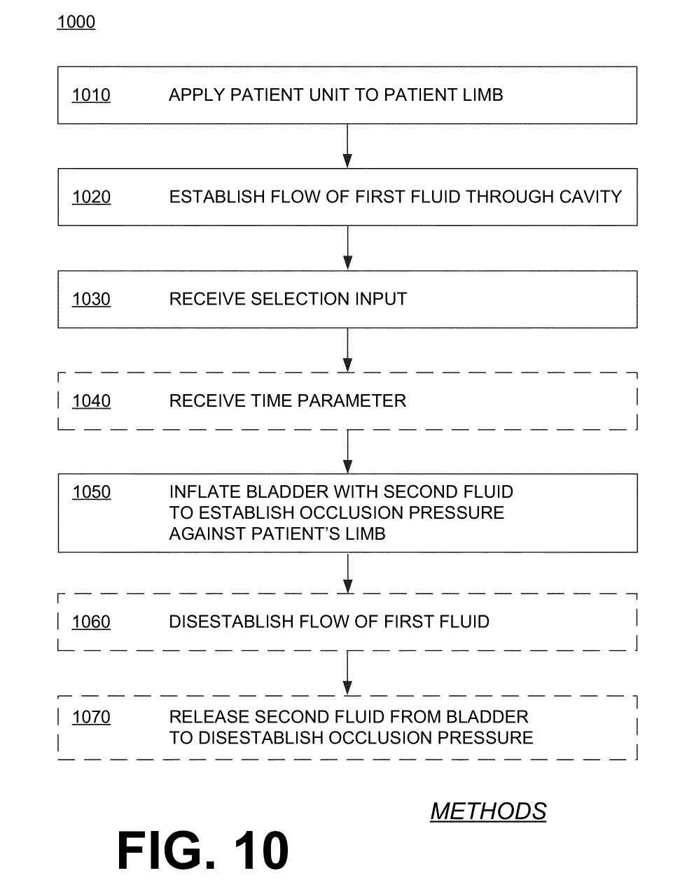

FIG. 10 shows a flowchart 1000 for describing methods according to embodiments. The methods of flowchart 1000 may also be practiced by embodiments described above.

According to an operation 1010, a patient unit is applied to a limb of patient. As above, the patient unit can include an inflatable bladder and a duct having a cavity, and it can be applied as shown in FIG. 1 and FIG. 2.

According to another, optional operation 1020, a flow of a first fluid can be established through the cavity. The flow can be established such that the first fluid exchanges heat with the patient's limb. An example of cooling is position “A” of FIG. 4 and FIG. 5.

According to another, optional operation 1030, a selection input SI is received. That can be equivalent to turning dial 420 of FIG. 4 from position “A” to position “B”. According to one more optional operation 1040, a time parameter is received.

According to another operation 1050, the bladder of the patient unit is inflated with a second fluid, responsive to the selection input. Inflating establishes an occlusion pressure against the patient's limb. As above, the occlusion pressure is large enough to substantially occlude blood circulation through the limb. Also, if a time parameter has also been received, the pressure is established according to the time parameter.

According to another, optional operation 1060, the flow of the first fluid through the cavity may become disestablished when the occlusion pressure is established. And according to another, optional operation 1070, then at least some of the second fluid is released from the bladder, so as to disestablish the occlusion pressure. The operation can alternate, and so the flow of the first fluid through the cavity can be disestablished when the occlusion pressure is established, and re-established when the occlusion pressure is disestablished.

According to another, optional operation, a blood pressure of the patient is measured non-invasively. A record of the measured blood pressure can be displayed, stored in memory, and so on.

In the methods described above, each operation can be performed as an affirmative step of doing, or causing to happen, what is written that can take place. Such doing or causing to happen can be by the whole system or device, or just one or more components of it. In addition, the order of operations is not constrained to what is shown, and different orders may be possible according to different embodiments. Moreover, in certain embodiments, new operations may be added, or individual operations may be modified or deleted. The added operations can be, for example, from what is mentioned while primarily describing a different system, device or method.

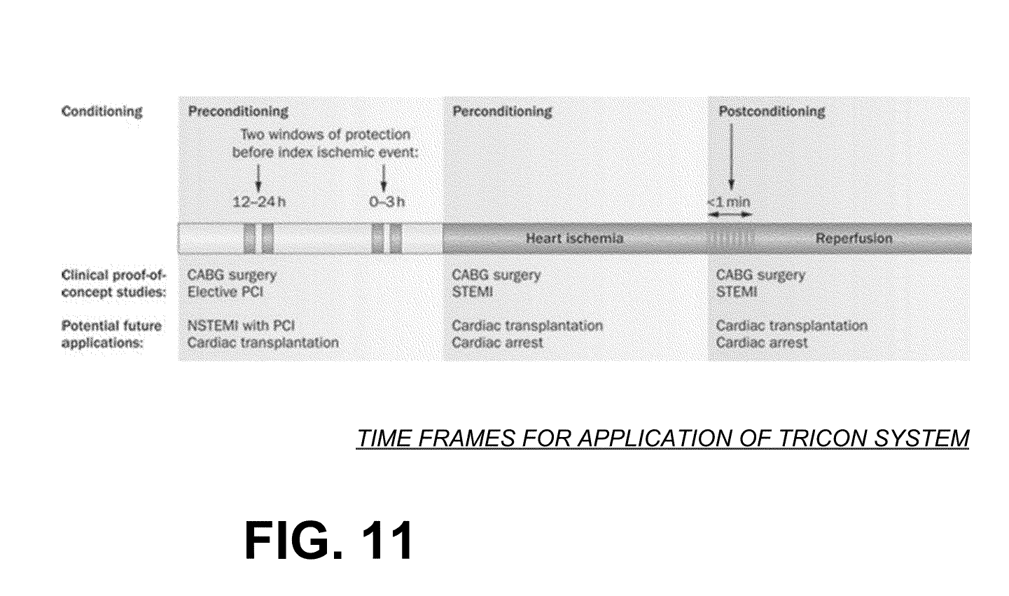

FIG. 11 is a table for showing different time frames, with reference to ischemic conditioning, for which TRICON system 100 can be applied. It will be appreciated that these include preconditioning, conditioning (which is also called preconditioning), and post conditioning. The diagram itself is from: Hausenloy, D. J. & Yellon, D. M. (2011) The therapeutic potential of ischemic conditioning: an update. Nat. Rev. Cardiol. doi:10.1038/nrcardio.2011.85, which is incorporated herein by reference.

This description includes one or more examples, but that does not limit how the invention may be practiced. Indeed, examples or embodiments of the invention may be practiced according to what is described, or yet differently, and also in conjunction with other present or future technologies.

Reference to any prior art in this specification is not, and should not be taken as, an acknowledgement or any form of suggestion that this prior art forms parts of the common general knowledge in any country.

A person skilled in the art will be able to practice the present invention in view of this description, which is to be taken as a whole. Details have been included to provide a thorough understanding. In other instances, well-known aspects have not been described, in order to not obscure unnecessarily the present invention.

Other embodiments include combinations and sub-combinations of features described herein, including for example, embodiments that are equivalent to: providing or applying a feature in a different order than in a described embodiment; extracting an individual feature from one embodiment and inserting such feature into another embodiment; removing one or more features from an embodiment; or both removing a feature from an embodiment and adding a feature extracted from another embodiment, while providing the advantages of the features incorporated in such combinations and sub-combinations.

The following claims define certain combinations and subcombinations of elements, features and steps or operations, which are regarded as novel and non-obvious. Additional claims for other such combinations and subcombinations may be presented in this or a related document.

What is claimed is:

1. A system, comprising:
a patient unit configured to be attached to a patient's limb, the patient unit including a flexible duct having a cavity and an inflatable bladder coupled to the duct;
a reservoir configured to contain a first fluid;
a circulation pump and at least one hose coupled to establish a circular flow of the first fluid from the reservoir through the cavity;
a compression pump and at least one hose coupled to inflate the bladder with a second fluid so as to establish pressure against the patient's limb;
an interface configured to receive a selection input; and
a controller configured to control the circulation pump and the compression pump to operate in at least a first mode or in a second mode according to the selection input, in which
in the first mode the circulation pump establishes the circular flow of the first fluid, and
in the second mode the pressure is an occlusion pressure that is large enough to substantially occlude blood circulation through the patient's limb.

2. The system of claim 1, in which
the patient unit is such that, when it is attached to the patient's limb, the duct contacts the limb and the bladder substantially surrounds the duct.

3. The system of claim 1, further comprising:
a memory;
a pressure gauge configured to measure the pressure; and
a sound sensor, and
in which a blood pressure of the patient is measured from an output of the pressure gauge at a time indicated by an output of the sound sensor and a record of the measured blood pressure is stored in the memory.

4. The system of claim 1, further comprising:
a temperature changing unit configured to change a temperature of the first fluid.

5. The system of claim 4, further comprising:
an auxiliary pump and at least one hose coupled to establish an auxiliary circular flow of the first fluid from the reservoir through the temperature changing unit.

6. The system of claim 5, in which the auxiliary circular flow bypasses the cavity.

7. The system of claim 1, further comprising:
a driver unit that contains the circulation pump and the compression pump.

8. The system of claim 1, further comprising:
a power supply configured to power the circulation pump and the compression pump.

9. The system of claim 1, in which
the interface is automatic, and configured to receive the selection input from another, cooperating device.

10. The system of claim 9, in which
a time parameter is received from the cooperating device, and
the pressure in the second mode is established according to the time parameter.

11. The system of claim 1, in which
the interface is a user interface, and configured to be operated by a rescuer for receiving the selection input.

12. The system of claim 1, in which
the patient unit further includes a bracket, and
the bladder, when inflated, establishes the pressure by being at least partly within the bracket and bracing against the bracket.

13. The system of claim 1, in which
in the first mode the pressure is nil.

14. The system of claim 1, in which
in the first mode the pressure is less than the occlusion pressure.

15. A controller of a system that includes a patient unit that is configured to be attached to a patient's limb, the patient unit including an inflatable bladder and a duct having a cavity, the system further including a circulation pump, a reservoir for a first fluid, a compression pump, hoses and an interface, the controller configured to:
cause the circulation pump to establish a flow of the first fluid through the cavity so that the first fluid exchanges heat with the patient's limb;
receive a selection input via the interface; and
responsive to the selection input, cause the compression pump to inflate the bladder with a second fluid so as to establish an occlusion pressure against the patient's limb, the occlusion pressure being large enough to substantially occlude blood circulation through the limb.

16. The controller of claim 15, further configured to:
receive a time parameter, and
in which the pressure is established according to the time parameter.

17. The controller of claim 15, in which
the flow of the first fluid through the cavity becomes disestablished when the occlusion pressure is established.

18. The controller of claim 17, in which
while the flow of the first fluid is established, the flow is also through a temperature changing unit, and
when the flow of the first fluid becomes disestablished, at least a portion of the first fluid is maintained through the temperature changing unit.

19. The controller of claim 15, further configured to:
then release at least some of the second fluid from the bladder so as to disestablish the occlusion pressure.

20. The controller of claim 19, in which
the flow of the first fluid through the cavity is disestablished when the occlusion pressure is established, and re-established when the occlusion pressure is disestablished.

21. A method for treating a patient, comprising:
applying a patient unit to a limb of the patient, the patient unit including an inflatable bladder and a duct having a cavity;
establishing a flow of a first fluid through the cavity so that the first fluid exchanges heat with the patient's limb;
receiving a selection input; and
responsive to the selection input, inflating the bladder with a second fluid so as to establish an occlusion pressure against the patient's limb, the occlusion pressure being large enough to substantially occlude blood circulation through the limb.

22. The method of claim 21, further comprising:
measuring a blood pressure of the patient; and
storing a record of the measured blood pressure in a memory.

23. The method of claim 21, further comprising:
receiving a time parameter, and
in which the pressure is established according to the time parameter.

24. The method of claim 21, further comprising:
measuring a first pressure of the patient.

25. The method of claim 21, in which
the flow of the first fluid through the cavity becomes disestablished when the occlusion pressure is established.

26. The method of claim 25, in which
while the flow of the first fluid is established, the flow is also through a temperature changing unit, and
when the flow of the first fluid becomes disestablished, at least a portion of the first fluid is maintained through the temperature changing unit.

27. The method of claim 21, further comprising:
then releasing at least some of the second fluid from the bladder so as to disestablish the occlusion pressure.

28. The method of claim 27, in which
the flow of the first fluid through the cavity is disestablished when the occlusion pressure is established, and re-established when the occlusion pressure is disestablished.
